(19) United States
(12) Patent Application Publication
(10) Pub. No.: US 2024/0299271 A1
(43) Pub. Date: Sep. 12, 2024

(54) TRANSPARENT CLEANSER

(71) Applicant: SHISEIDO COMPANY, LTD., Tokyo (JP)

(72) Inventors: Yuki WATANABE, Tokyo (JP); Hiromi TANAKA, Tokyo (JP); Yukiko HIRUMA, Tokyo (JP)

(73) Assignee: SHISEIDO COPMAPY, LTD., Tokyo (JP)

(21) Appl. No.: 18/563,710

(22) PCT Filed: Jun. 14, 2022
(86) PCT No.: PCT/JP2022/023761

§ 371 Date: Nov. 22, 2023

(30) Foreign Application Priority Data
Jun. 23, 2021 (JP) 2021-104413

(51) Int. Cl. A61K 8/37; A61Q 1/14; A61Q 19/10

(52) U.S. Cl. CPC A61K 8/375; A61Q 1/14; A61Q 19/10; A61K 2800/596

(57) ABSTRACT

A transparent cleanser including: (A) an ester oil; (B) an aliphatic hydrocarbon having 8 or more and 20 or less carbon atoms; and (C) a nonionic surfactant, in which a content of the ester oil (A) is 40% by mass or more and 90% by mass or less to a total amount, the nonionic surfactant (C) includes: (C1) a polyglyceryl difatty acid ester consisting of a fatty acid having 8 or more and 10 or less carbon atoms and a polyglyceryl having an average polymerization degree of 4 or more and 10 or less; and (C2) a polyglyceryl difatty acid ester consisting of a fatty acid having 18 or more and 22 or less carbon atoms and a polyglyceryl having an average polymerization degree of 8 or more and 15 or less, and a mass ratio of the components (C1) and (C2) is from 2.5:1 to 10:1.

### BACKGROUND OF THE INVENTION

### Technical Field

The present invention relates to a transparent cleanser. More particularly, it relates to a transparent cleanser for use in removing makeup.

### Background Art

The primary purpose of makeup-removing cleansers is to remove makeup cosmetics such as mascara, eye shadow, lipstick, and foundation. When taking a bath after going out, makeup removal is often done with face washing. However, conventional makeup-removing cleansers were problematic in that, for example, when used with wet hands or face in the bathroom, a makeup-removing cleanser is emulsified as a result of water mixed therewith, resulting in poor appearance because of white turbidness, or the emulsification causes the makeup-removing effect of the makeup-removing cleanser to deteriorate significantly.

To solve the above-described problem, a cleanser has been proposed that does not reduce its makeup-removing effect even when used with wet hands. For example, Patent Literature 1 suggests an oil-based liquid skin cleansing composition comprising: (A) ester oil; (B) a nonionic surfactant having an HLB value of from 8 to less than 12; (C) at least one or two members having an HLB value of 12 or more selected from polyglyceryl lauryl ether, polyglyceryl dicaprate, polyglyceryl myristate, polyglyceryl tricaprylate, and polyglyceryl diisostearate. Patent Literature 2 suggests a transparent liquid cleansing cosmetic containing: (A) monoglycerin fatty acid ester having a fatty acid residue having from 12 to 18 carbon atoms; (B) polyglycerin fatty acid ester having a fatty acid residue having from 12 to 18 carbon atoms; (C) a water-soluble nonionic surfactant other than the components (A) and (B); (D) an oil-based component that is liquid at 25° C.; and (E) 1% to 12% by weight of water, in which the ratio (A): (B) is in a range of from 20:80 to 80:20 (weight ratio), and white turbidness is not seen when 50 parts by weight of water is added to 100 parts by weight of the composition. Patent Literature 3 suggests an oil-based cleansing cosmetic containing: (A) a polyglycerin difatty acid ester consisting of a fatty acid having from 8 to 10 carbon atoms and a polyglycerin having an average polymerization degree of from 4 to 10; (B) an esterified polyglycerin difatty acid ester consisting of a fatty acid having from 18 to 22 carbon atoms and a polyglycerin having an average polymerization degree of from 8 to 15; and (C) an oiling agent, characterized in that the ratio (A): (B) is from 12:5 to 1:1. However, there is still a need for further improvements in properties such as makeup removal effects over conventional makeup-removing cleansers.

### CITATION LIST

### Patent Literature

[Patent Literature 1] JP 2020-40918 A
[Patent Literature 2] Japanese Patent No. 3964760
[Patent Literature 3] Japanese Patent No. 6684648

### SUMMARY OF INVENTION

### Technical Problem

An object of the present invention is to provide a transparent cleanser that has an excellent makeup-removing effect even when used with wet hands.

As a result of intensive studies to solve the above-described problem, the present inventors found that the above-described problem can be solved by mixing an ester oil, an aliphatic hydrocarbon having 8 or more and 20 or less carbon atoms, and a nonionic surfactant in a transparent cleanser and adjusting the ratio of two specific polyglyceryl difatty acid esters serving as nonionic surfactants. This has led to the completion of the present invention.

### Solution to Problem

According to the present invention, the following inventions are provided.

[1] A transparent cleanser, comprising:
(A) an ester oil;
(B) an aliphatic hydrocarbon having 8 or more and 20 or less carbon atoms; and
(C) a nonionic surfactant,
   wherein a content of the ester oil (A) is 40% by mass or more and 90% by mass or less with respect to a total amount of the transparent cleanser,
   wherein the nonionic surfactant (C) comprises:
(C1) a polyglyceryl difatty acid ester consisting of a fatty acid having 8 or more and 10 or less carbon atoms and a polyglyceryl having an average polymerization degree of 4 or more and 10 or less; and
(C2) a polyglyceryl difatty acid ester consisting of a fatty acid having 18 or more and 22 or less carbon atoms and a polyglyceryl having an average polymerization degree of 8 or more and 15 or less, and wherein a mass ratio of the components (C1) and (C2) is from 2.5:1 to 10:1.

[2] The transparent cleanser according to [1], wherein the component (B) is at least one selected from the group consisting of hydrogenated polyisobutene having 16 or less carbon atoms, isododecane, and isohexadecane.

[3] The transparent cleanser according to [1] or [2], wherein a content of the component (B) is 3% by mass or more and 30% by mass or less with respect to the total amount of the transparent cleanser.

[4] The transparent cleanser according to any one of [1] to [3], wherein a total content of the components (C1) and (C2) is 8% by mass or more and 45% by mass or less with respect to the total amount of the transparent cleanser.

[5] The transparent cleanser according to any one of [1] to [4], wherein the component (C1) is at least one selected from the group consisting of hexaglyceryl dicaprylate and hexaglyceryl dicaprate.

[6] The transparent cleanser according to any one of [1] to [5], wherein the component (C2) is at least one selected from the group consisting of decaglyceryl diisostearate and decaglyceryl dioleate.

[7] The transparent cleanser according to any one of [1] to [6], which has a moisture content of 5% by mass or less.

[8] The transparent cleanser according to any one of [1] to [7], which further comprises a moisturizer.

[9] The transparent cleanser according to any one of [1] to [8], which has a viscosity of 5000 mPa-s or less when adding 30 parts by mass of water to 100 parts by mass of the transparent cleanser.

[10] The transparent cleanser according to any one of [1] to [9], which is a skin cleanser.

[11] The transparent cleanser according to any one of [1] to [9], which is a cleansing cosmetic.

### Advantageous Effects of Invention

According to the present invention, a transparent cleanser that has an excellent makeup-removing effect even when used with wet hands is provided.

### DESCRIPTION OF EMBODIMENTS

### [Transparent Cleanser]

The transparent cleanser according to the present invention comprises: (A) an ester oil; (B) an aliphatic hydrocarbon having 8 or more and 20 or less carbon atoms; and (C) a nonionic surfactant, which are as essential components. The transparent cleanser may further comprise a moisturizer, water, and other components depending on the purpose. The transparent cleanser has an excellent makeup-removing effect even when used with wet hands and thus can be used appropriately as a skin cleanser or a cleansing cosmetic. The term “transparent” used in the present invention means no turbidness, such as white turbidness. The transparent cleanser may be translucent, but preferably has high transparency. Each component contained in the transparent cleanser will be explained in detail below.

### ((A) Ester Oil)

The ester oil is not particularly limited as long as it is an ester oil commonly used as a material for cleansers. Examples of an ester oil include isopropyl myristate, cetyl ethylhexanoate, octyldodecyl myristate, isopropyl palmitate, butyl stearate, hexyl laurate, myristyl myristate, decyl oleate, hexyldecyl dimethyloctoate, cetyl lactate, myristyl lactate, lanolin acetate, isocetyl stearate, isocetyl isostearate, cholesteryl 12-hydroxystearate, ethylene glycol di-2-ethylhexanoate, dipentaerythritol fatty acid ester, N-alkyl glycol monoisostearate, neopentyl glycol dicaprate, diisostearyl malate, glycerin di-2-heptylundecanoate, trimethylolpropane tri-2-ethylhexanoate, trimethylolpropane triisostearate, pentaerythritol tetra-2-ethylhexanoate, glycerin tri-2-ethylhexanoate, glycerin trioctanoate, glycerin triisopalmitate, trimethylolpropane triisostearate, cetyl 2-ethylhexanoate, 2-ethylhexyl palmitate, glyceryl trimyristate, Tri-2-heptylundecanoic acid glyceride, castor oil fatty acid methyl ester, oleyl oleate, acetoglyceride, 2-heptylundecyl palmitate, diisobutyl adipate, N-lauroyl-L-glutamic acid-2-octyldodecyl ester, di-2-heptylundecyl adipate, ethyl laurate, di-2-ethylhexyl sebacate, 2-hexyldecyl myristate, 2-hexyldecyl palmitate, 2-hexyldecyl adipate, diisopropyl sebacate, 2-ethylhexyl succinate, and triethyl citrate. These may be used singly or in a combination of two or more.

The content of the ester oil is 40% by mass or more and 90% by mass or less, preferably 55% by mass or more and 85% by mass or less, more preferably 60% by mass or more and 80% by mass or less with respect to the total amount of the transparent cleanser. As long as the content of the ester oil is in the above-described numerical range, even when the transparent cleanser is used with wet hands, high transparency and improved makeup-removing effects can be obtained.

### ((B) Aliphatic Hydrocarbon Having 8 or More and 20 or Less Carbon Atoms)

The aliphatic hydrocarbon having 8 or more and 20 or less carbon atoms is preferably a saturated aliphatic hydrocarbon having preferably 10 or more and 18 or less carbon atoms, more preferably 12 or more and 16 or less carbon atoms. Examples of the saturated aliphatic hydrocarbon having 8 or more and 20 or less carbon atoms include hydrogenated polyisobutene having 16 or less carbon atoms, isododecane, and isohexadecane. These may be used singly or in a combination of two or more.

The content of the aliphatic hydrocarbon having 8 or more and 20 or less carbon atoms is preferably 3% by mass or more and 30% by mass or less, more preferably 4% by mass or more and 20% by mass or less, still more preferably 5% by mass or more and 15% by mass or less with respect to the total amount of the transparent cleanser. As long as the content of the aliphatic hydrocarbon having 8 or more and 20 or less carbon atoms is in the above-described numerical range, even when the transparent cleanser is used with wet hands, high transparency and improved makeup-removing effects can be obtained.

### ((C) Nonionic Surfactant)

The nonionic surfactant comprises: the following components (C1) and (C2).

It is preferable to use a nonionic surfactant having an HLB value of 8 or more and 12 or less as a nonionic surfactant. The HLB value is an index indicating the hydrophilic-lipophilic balance, and in the present invention, the value calculated using the following equation according to the Griffin method is used.

HLB value=20×(1−Saponification value of ester/Neutralization value of fatty acid)

### ((C1) Polyglyceryl Difatty Acid Ester Consisting of Fatty Acid Having 8 or More and 10 or Less Carbon Atoms and Polyglyceryl Having Average Polymerization Degree of 4 or More and 10 or Less)

Examples of the fatty acid having 8 or more and 10 or less carbon atoms include caprylic acid and capric acid. The average polymerization degree of the polyglyceryl is preferably 6 or more and 8 or less. Specific examples of the polyglyceryl difatty acid ester consisting of a fatty acid having 8 or more and 10 or less carbon atoms and a polyglyceryl having an average polymerization degree of 4 or more and 10 or less include hexaglyceryl dicaprylate and hexaglyceryl dicaprate. These may be used singly or in a combination of two or more.

### ((C2) Polyglyceryl Difatty Acid Ester Consisting of Fatty Acid Having 18 or More and 22 or Less Carbon Atoms and Polyglyceryl Having Average Polymerization Degree of 8 or More and 15 or Less)

Examples of the fatty acid having 18 or more and 22 or less carbon atoms include stearic acid, isostearic acid, oleic acid, linoleic acid, arachidic acid, and behenic acid. The average polymerization degree of the polyglyceryl is preferably 10 or more and 12 or less. Specific examples of the polyglyceryl difatty acid ester consisting of a fatty acid having 18 or more and 22 or less carbon atoms and a polyglyceryl having an average polymerization degree of 8 or more and 15 or less include decaglyceryl diisostearate and decaglyceryl dioleate. These may be used singly or in a combination of two or more.

The mass ratio of the components (C1) and (C2) in the transparent cleanser is from 2.5:1 to 10:1, preferably 3.0:1 to 9.0:1. As long as the mass ratio of the components (C1) and (C2) is in the above-described range, even when the transparent cleanser is used with wet hands, high transparency and excellent makeup-removing effects can be obtained.

The total content of the components (C1) and (C2) is preferably 8% by mass or more and 45% by mass or less, more preferably 10% by mass or more and 35% or less by mass with respect to the total amount of the transparent cleanser. As long as the content of the nonionic surfactant having an HLB value of 8 or more and 12 or less is in the above-described numerical range, even when the transparent cleanser is used with wet hands, high transparency and improved makeup-removing effects can be obtained.

### (Moisturizer)

The moisturizer is not particularly limited, and any moisturizer commonly used as a material for cleansers can be used. Examples of a moisturizer include polyethylene glycol, propylene glycol, glycerin, diglycerin, 1,3-butylene glycol, xylitol, sorbitol, maltitol, chondroitin sulfate, hyaluronic acid, mucoitin sulfate, caronic acid, atelocollagen, sodium lactate, bile salts, dl-pyrrolidone carboxylate, short chain soluble collagen, diglycerin (EO) PO adduct, Rosa roxburghii extract, yarrow extract, and melilot extract. These may be used singly or in a combination of two or more. In particular, it is preferable to use glycerin.

The content of the moisturizer is preferably 0.1% by mass or more and 10% by mass or less, more preferably 0.5% by mass or more and 5% by mass or less with respect to the total amount of the transparent cleanser.

### (Water)

The water is not particularly limited, but water used for cosmetics, quasi-drugs, and the like can be used; for example, purified water, ion-exchanged water, tap water, and the like can be used.

The content of water can be adjusted as appropriate depending on the contents of components other than water. For example, the content of water is preferably 5% by mass or less, more preferably 3% by mass or less, still more preferably 1% by mass or less with respect to the total amount of the cleanser.

### (Other Components)

The transparent cleanser according to the present invention can contain, in addition to the above-described components, components commonly used in cleansers for cosmetics and pharmaceuticals and is produced according to a conventional method. Examples of the other components include the following. The other components can be produced by further compounding one or more of the following components as long as the effects of the present invention are achieved.

Examples of a powder component include: inorganic powders (e.g., talc, kaolin, mica, sericite, muscovite, phlogopite, synthetic mica, lepidolite, biotite, vermiculite, magnesium carbonate, calcium carbonate, aluminum silicate, barium silicate, calcium silicate, magnesium silicate, strontium silicate, tungstate metal salts, magnesium, silica, zeolite, barium sulfate, calcined calcium sulfate (calcined gypsum), calcium phosphate, fluoroapatite, hydroxyapatite, ceramic powder, metal soaps (e.g., zinc myristate, calcium palmitate, and aluminum stearate), and boron nitride); organic powders (e.g., polyamide resin powder (nylon powder), polyethylene powder, polymethyl methacrylate powder, polystyrene powder, copolymer resin powder of styrene and acrylic acid, benzoguanamine resin powder, polytetrafluoroethylene powder, and cellulose powder); inorganic white pigments (e.g., titanium dioxide, zinc oxide, etc.); inorganic red pigments (e.g., iron oxide (colcothar) and iron titanate); inorganic brown pigments (e.g., γ-iron oxide); inorganic yellow pigments (e.g., yellow iron oxide and yellow clay); inorganic black pigments (e.g., black iron oxide and lower titanium oxide); inorganic purple pigments (e.g., mango violet and cobalt violet); inorganic green pigments (e.g., chromium oxide, chromium hydroxide, and cobalt titanate); inorganic blue pigments (e.g., ultramarine and navy blue); pearl pigments (e.g., titanium-oxide-coated mica, titanium-oxide-coated bismuth oxychloride, titanium-oxide-coated talc, colored titanium-oxide-coated mica, bismuth oxychloride, and fish scale foil); metal powder pigments (e.g., aluminum powder and copper powder); organic pigments such as zirconium, barium, and aluminum lake (e.g.: organic pigments such as Red No. 201, Red No. 202, Red No. 204, Red No. 205, Red No. 220, Red No. 226, Red No. 228, Red No. 405, Orange No. 203, Orange No. 204, Yellow No. 205, Yellow No. 401, and Blue No. 404; Red No. 3, Red No. 104, Red No. 106, Red No. 227, Red No. 230, Red No. 401, Red No. 505, Orange No. 205, Yellow No. 4, Yellow No. 5, Yellow No. 202, Yellow No. 203, Green No. 3, and Blue No. 1); and natural pigments (e.g., chlorophyll and β-carotene).

Examples of liquid fats and oils include avocado oil, camellia oil, turtle oil, macadamia nut oil, corn oil, mink oil, Olive oil, rapeseed oil, egg yolk oil, sesame oil, persic oil, wheat germ oil, sasanqua oil, castor oil, linseed oil, safflower oil, cottonseed oil, perilla oil, soybean oil, peanut oil, teaseed oil, Kaya nut oil, rice bran oil, Paulownia fargesii Franch. oil, Japanese tung oil, jojoba oil, germ oil, and triglycerin.

Examples of solid fats and oils include cocoa butter, coconut oil, horse tallow, hydrogenated coconut oil, palm oil, beef tallow, mutton tallow, hydrogenated beef tallow, palm kernel oil, pork fat, beef bone fat, Rhus succedanea fruit kernel oil, hydrogenated oil, beef leg fat, Rhus succedanea fruit wax, and hydrogenated castor oil.

Examples of waxes include beeswax, candelilla wax, cotton wax, carnauba wax, bayberry wax, Chinese wax, whale wax, montan wax, rice bran wax, lanolin, kapok (Schefflera arboricola) wax, lanolin acetate, liquid lanolin, sugar cane wax, isopropyl lanolin fatty acid, hexyl laurate, reduced lanolin, jojoba wax, hard lanolin, shellac wax, POE lanolin alcohol ether, POE lanolin alcohol acetate, POE cholesterol ether, lanolin fatty acid polyethylene glycol, and POE hydrogenated lanolin alcohol ether.

Examples of higher alcohols include linear chain alcohols (e.g., lauryl alcohol, cetyl alcohol, stearyl alcohol, behenyl alcohol, myristyl alcohol, oleyl alcohol, and cetostearyl alcohol); and branched chain alcohols (e.g., monostearyl glycerin ether (batyl alcohol), 2-decyltetradecinol, lanolin alcohol, cholesterol, phytosterol, hexyldodecanol, isostearyl alcohol, and octyldodecanol).

Examples of silicone oils include chain polysiloxanes (e.g., dimethylpolysiloxane, methylphenylpolysiloxane, and diphenylpolysiloxane), cyclic polysiloxanes (e.g., octamethylcyclotetrasiloxane, decamethylcyclopentasiloxane, and dodecamethylcyclohexasiloxane), silicone resin forming a three-dimensional network structure, silicone rubber, and various modified polysiloxanes (e.g., amino-modified polysiloxane, polyether-modified polysiloxane, alkyl-modified polysiloxane, and fluorine-modified polysiloxane).

Examples of natural water-soluble polymers include: plant-based polymers (e.g., gum arabic, gum tragacanth, galactan, guar gum, carob gum, gum karaya, carrageenan, pectin, agar, quince seed (marmelo), algae colloid (seaweed extract), starch (rice, corn, potato, wheat), and glycyrrhizic acid); microbial polymers (e.g., xanthan gum, dextran, succinoglucan, and pullulan); and animal-based polymers (e.g., collagen, casein, albumin, and gelatin).

Examples of semi-synthetic water-soluble polymers include starch-based polymers (e.g., carboxymethyl starch and methylhydroxypropyl starch); cellulose polymers (e.g., methylcellulose, ethylcellulose, methylhydroxypropylcellulose, hydroxyethylcellulose, sodium cellulose sulfate, hydroxypropylcellulose, carboxymethylcellulose, sodium carboxymethylcellulose, crystalline cellulose, and cellulose powder); and alginate polymers (e.g., sodium alginate and propylene glycol alginate).

Examples of synthetic water-soluble polymers include vinyl polymers (e.g., polyvinyl alcohol, polyvinyl methyl ether, polyvinylpyrrolidone, and carboxyvinyl polymer); polyoxyethylene polymers (e.g., polyoxyethylene polyoxypropylene copolymers of polyethylene glycol 20,000, 40,000, and 60,000); acrylic polymers (e.g., sodium polyacrylate, polyethyl acrylate, and polyacrylamide); polyethyleneimine; and cationic polymer.

Examples of thickeners include gum arabic, carrageenan, gum karaya, gum tragacanth, carob gum, quince seed (marmelo), casein, dextrin, gelatin, sodium pectate, sodium alaginate, methylcellulose, ethylcellulose, CMC, hydroxyethyl cellulose, hydroxypropyl cellulose, PVA, PVM, PVP, sodium polyacrylate, carboxyvinyl polymer, locust bean gum, guar gum, tamarind gum, dialkyldimethylammonium cellulose sulfate, xanthan gum, magnesium aluminum silicate, bentonite, hectorite, silicic acid A1Mg (vegum), laponite, and silicic anhydride.

Examples of UV absorbers include benzoic acid-based UV absorbers (e.g., para-aminobenzoic acid (hereinafter, abbreviated as PABA), PABA monoglycerin ester, N,N-dipropoxy PABA ethyl ester, N,N-diethoxy PABA ethyl ester, N,N-dimethyl PABA ethyl ester, N,N-dimethyl PABA butyl ester, and N,N-dimethyl PABA ethyl ester); anthranilic acid-based UV absorbers (e.g., homomenthyl-N-acetylanthranilate); salicylic acid-based UV absorbers (e.g., amyl salicylate, menthyl salicylate, homomenthyl salicylate, octyl salicylate, phenyl salicylate, benzyl salicylate, and p-isopropanolphenyl salicylate); cinnamic acid-based UV absorbers (e.g., octyl cinnamate, ethyl-4-isopropyl cinnamate, methyl-2,5-diisopropyl cinnamate, ethyl-2,4-diisopropyl cinnamate, methyl-2,4-diisopropyl cinnamate, propyl-p-methoxy cinnamate, isopropyl-p-methoxy cinnamate, isoamyl-p-methoxy cinnamate, octyl-p-methoxy cinnamate (2-ethylhexyl-p-methoxy cinnamate), 2-ethoxyethyl-p-methoxy cinnamate, cyclohexyl-p-methoxycinnamate, ethyl-α-cyano-β-phenyl cinnamate, 2-ethylhexyl-α-cyano-β-phenyl cinnamate, and glyceryl mono-2-ethylhexanoyl-diparamethoxy cinnamate); benzophenone-based UV absorbers (e.g., 2,4-dihydroxybenzophenone, 2,2′-dihydroxy-4-methoxybenzophenone, 2,2′-dihydroxy-4,4′-dimethoxybenzophenone, 2,2′,4,4′-tetrahydroxybenzophenone, 2-hydroxy-4-methoxybenzophenone, 2-hydroxy-4-methoxy-4′-methylbenzophenone, 2-hydroxy-4-methoxybenzophenone-5-sulfonic acid salt, 4-phenylbenzophenone, 2-ethylhexyl-4′-phenyl-benzophenone-2-carboxylate, 2-hydroxy-4-n-octoxybenzophenone, and 4-hydroxy-3-carboxybenzophenone); 3-(4′-methylbenzylidene)-d,1-camphor, 3-benzylidene-d,1-camphor; 2-phenyl-5-methylbenzoxazole; 2,2′-hydroxy-5-methylphenylbenzotriazole; 2-(2′-hydroxy-5′-t-octylphenyl)benzotriazole; 2-(2′-hydroxy-5′-methylphenylbenzotriazole; dibenzalazine; dianisoylmethane; 4-methoxy-4′-t-butyldibenzoylmethane; and 5-(3,3-dimethyl-2-norbornylidene)-3-pentane-2-on).

Examples of metal ion sequestering agents include 1-hydroxyethane-1,1-diphosphonic acid, 1-hydroxyethane-1,1-diphosphonic acid tetrasodium salt, disodium edetate, trisodium edetate, tetrasodium edetate, sodium citrate, sodium polyphosphate, sodium metaphosphate, gluconic acid, phosphoric acid, citric acid, ascorbic acid, succinic acid, edetic acid, and trisodium ethylenediamine hydroxyethyl triacetate.

Examples of lower alcohols include ethanol, propanol, isopropanol, isobutyl alcohol, and t-butyl alcohol.

Examples of monosaccharides include three-carbon sugars (trioses) (e.g., D-glycerylaldehyde and dihydroxyacetone); four-carbon sugars (tetroses) (e.g., D-erythrose, D-erythrulose, D-threose, and erythritol); five-carbon sugars (pentoses) (e.g., L-arabinose, D-xylose, L-lyxose, D-arabinose, D-ribose, D-ribulose, D-xylulose, and L-xylulose); six-carbon sugars (hexoses) (e.g., D-glucose, D-talose, D-bucicose, D-galactose, D-fructose, L-galactose, L-mannose, and D-tagatose); seven-carbon sugars (heptoses) (e.g., aldoheptose and heprose); eight-carbon sugars (octoses) (e.g., octulose); deoxy sugars (e.g., 2-deoxy-D-ribose, 6-deoxy-L-galactose, and 6-deoxy-L-mannose); amino sugars (e.g., D-glucosamine, D-galactosamine, sialic acid, aminouronic acid, and muramic acid); and uronic acids (e.g., D-glucuronic acid, D-mannuronic acid, L-guluronic acid, D-galacturonic acid, and L-iduronic acid).

Examples of oligosaccharides include sucrose, gentianose, umbelliferose, lactose, planteose, isolychnoses, α,α-trehalose, raffinose, lychnoses, umbilicin, stachyose, and verbascoses.

Examples of amino acids include neutral amino acids (e.g., threonine and cysteine); and basic amino acids (e.g., hydroxylysine). In addition, examples of amino acid derivatives include sodium acyl sarcosine (sodium lauroyl sarcosine), acyl glutamate, sodium acyl β-alanine, glutathione, and pyrrolidone carboxylic acid.

Examples of organic amines include monoethanolamine, diethanolamine, triethanolamine, morpholine, triisopropanolamine, 2-amino-2-methyl-1,3-propanediol, and 2-amino-2-methyl-1-propanol.

Examples of the polymer emulsion include acrylic resin emulsion, polyethyl acrylate emulsion, acrylic resin liquid, polyacrylalkyl ester emulsion, polyvinyl acetate resin emulsion, and natural rubber latex.

Examples of pH adjusters include buffers such as lactic acid-sodium lactate, citric acid-sodium citrate, and succinic acid-sodium succinate.

Examples of vitamins include vitamins A, B1, B2, B6, C, E, and derivatives thereof, pantothenic acid and derivatives thereof, and biotin.

Examples of antioxidants include tocopherols, dibutylhydroxytoluene, butylhydroxyanisole, gallic acid esters, sulfites, and bisulfites. Examples of antioxidant aids include phosphoric acid, citric acid, ascorbic acid, maleic acid, malonic acid, succinic acid, fumaric acid, cephalin, hexametaphosphate, phytic acid, and ethylenediaminetetraacetic acid.

Examples of other compoundable components include preservatives (e.g., ethyl paraben and butyl paraben); anti-inflammatory agents (e.g., glycyrrhizic acid derivatives, glycyrrhetinic acid derivatives, salicylic acid derivatives, hinokitiol, zinc oxide, and allantoin); whitening agents (e.g., placenta extract, saxifraga extract, and arbutin); various extracts (e.g., phellodendron bark, coptis rhizome, lithospermum root, peony root, swertia herb, birch, sage, loquat, ginseng, aloe, tree mallow, iris, grape, coix seed, luffa, lily, saffron, cnidium rhizome, ginger, hypericum, ononis, garlic, capsicum, citrus unshiu peel, Angelica acutiloba, and seaweed), excipients (e.g., royal jelly, photosensitive elements, and cholesterol derivatives); blood circulation promoters (e.g., 4-hydroxy-3-methoxybenzyl nonylic acid amide, nicotinic acid benzyl ester, nicotinic acid β-butoxyethyl ester, capsaicin, zingerone, cantharides tincture, ichthammol, tannic acid, α-borneol, tocopherol nicotinate, inositol hexanicotinate, cyclandelate, cinnarizine, tolazoline, acetylcholine, verapamil, cepharanthine, and γ-oryzanol); antiseborrheic agents (e.g., sulfur and thianthol); and anti-inflammatory agents (e.g., tranexamic acid, thiotaurine, and hypotaurine).

For the transparent cleanser according to the present invention, the viscosity when adding 30 parts by mass of water to 100 parts by mass of the transparent cleanser is preferably 5000 mPa-s or less, more preferably 3000 mPa-s or less, still more preferably 1000 mPa-s or less, furthermore preferably 500 mPa-s or less. It is generally 1 mPa-s or more and may be 10 mPa-s or more. The viscosity at 30° C. is a value measured using a commercially available BH-type viscometer at a rotation speed of 12 rpm. The rotor for measurement can be appropriately selected depending on the viscosity.

### EXAMPLES

The present invention will be specifically explained based on the following examples, but the present invention is not limited to these examples.

The compounding amounts are expressed in parts by mass unless otherwise specified.

### Examples 1 to 17, Comparative Examples 1 to 9

### <Preparation of Cleanser>

Cleansers for the Examples and Comparative Examples were prepared using the formulations listed in Tables 1 to 4. In Tables 1 to 4, the compounding ratio of each component is in parts by mass.

### <Evaluation of Cleanser>

Assuming that the cleansers prepared above would be used with wet hands, a mixed solution was prepared by adding 30 parts by mass of water to 100 parts by mass of each cleanser (hereinafter referred to as “30% water-mixed cleanser”). Each 30% water-mixed cleanser was evaluated in the following manner.

[Makeup Removal with 30% Water-Mixed Cleanser]

### (Evaluation Method)

An expert panel of three women in their 30s and 40s, who have received training in sensory evaluation and can perform evaluations based on specific criteria, were selected. The expert panel evaluated the effect of removing makeup (mascara and lipstick) using each 30% water-mixed cleanser for removing makeup. The 30% water-mixed cleansers were evaluated on a five-level scale using the following absolute evaluation for giving scores. The average value was calculated from the total evaluation scores of all the panel members for each 30% water-mixed cleanser for judgment. The evaluation results are shown in Tables 1 to 4. If the evaluation result is A or B, a pass is given.

### <Evaluation Points>

5 points: Had a remarkably excellent high makeup-removing effect.
4 points: Had an excellent makeup-removing effect.
3 points: Had an average makeup-removing effect.
2 points: Had a poor makeup-removing effect.
1 point: Had a remarkably poor makeup-removing effect.

### <Evaluation Criteria>

A: Average score of 4.5 points or more
B: Average score of 3.5 points or more and less than 4.5 points
C: Average score of 2.5 points or more and less than 3.5 points
D: Average score of less than 2.5 points

### [Appearance of 30% Water-Mixed Cleanser]

### <Evaluation Method>

Each 30% water-mixed cleanser in an amount of 1 g was placed in a transparent glass petri dish having a diameter of 30 mm, and it was confirmed whether the 10-point type could be read with the naked eye through the cleanser when viewed from above. The evaluation results are shown in Tables 1 to 4. If the evaluation result is A or B, a pass is given.

### <Evaluation Criteria>

A: It was easily readable.
B: It was readable.
C: It was not readable.

### [Viscosity of 30% Water-Mixed Cleanser]

The viscosity (mPa-s) of each 30% water-mixed cleanser at 30° C. was measured using a BH-type viscometer (model name: TVB-15M; Rotor No. 2 at a viscosity of less than 2500 mPa-s, Rotor No. 3 at a viscosity of 2500 mPa-s or more and less than 10000 mPa-s, Rotor No. 4 at a viscosity of 10000 mPa-s or more) at a rotation speed of 12 rpm and evaluated based on the following criteria. The measurement results and evaluation results are shown in Tables 1 to 4. If the evaluation result is A or B, a pass is given.

### <Evaluation Criteria>

A: Even when 30% by mass of water was mixed, the viscosity did not increase, resulting in a range of 500 mPa-s or less.

B: When 30% by mass of water was mixed, the viscosity slightly increased, resulting in a range of more than 500 mPa-s and 5000 mPa-s or less.

C: When 30% by mass of water was mixed, the viscosity increased, resulting in a range of more than 5000 mPa-s and 50000 mPa-s or less.

D: When 30% by mass of water was mixed, the viscosity significantly increased, resulting in a range of more than 50000 mPa-s.

TABLE 1

| | | Example | Example | Example | Example | Example | Example |
|---|---|---|---|---|---|---|---|
| Component | | 1 | 2 | 3 | 4 | 5 | 6 |
| A | Cetyl ethylhexanoate (CIO) | 48.0 | 53.5 | 60.0 | 40.0 | 40.0 | 40.0 |
| | Isopropyl myristate (IPMR) | 7.0 | 7.58 | 13.5 | 42.3 | 43.5 | 46.8 |
| B | Hydrogenated polyisobutene | 5.0 | 8.92 | 5.0 | 5.0 | 5.0 | 5.0 |
| C | (C1) Polyglyceryl-6 dicaprate*¹ | 30 | 22.5 | 15 | 8.4 | 7.5 | 6.0 |
| | (C2) Polyglyceryl-10 geoleate*² | 10 | 7.5 | 5.0 | 2.8 | 2.5 | 2.0 |
| | Water | 0 | 0 | 0.5 | 0.5 | 0.5 | 0.2 |
| | Glycerin | 0 | 0 | 1.0 | 1.0 | 1.0 | 0 |
| | Total (parts by mass) | 100 | 100 | 100 | 100 | 100 | 100 |
| | (C1)/(C2) (mass ratio) | 3.0 | 3.0 | 3.0 | 3.0 | 3.0 | 3.0 |
| Evaluation | Makeup removal with 30% | A | A | A | A | A | A |
| | water-mixed cleanser | | | | | | |
| | Appearance of 30% water-mixed | A | A | A | A | A | B |
| | cleanser | | | | | | |
| | Viscosity of 30% water-mixed | A | A | A | A | A | A |
| | cleanser | | | | | | |

*¹Hexaglyceryl dicaprylate consisting of capric acid (having 10 carbon atoms) and polyglyceryl having an average polymerization degree of 6 (HLB value: 10.2)
*²Decaglyceryl dioleate consisting of oleic acid (having 18 carbon atoms) and polyglyceryl having an average polymerization degree of 10 (HLB value: 11.9)

TABLE 2

| Component | | Example 7 | Example 8 | Example 9 | Example 10 | Example 11 |
|---|---|---|---|---|---|---|
| A | Cetyl ethylhexanoate (CIO) | 40.0 | 60.0 | 60.0 | 58.5 | 60.0 |
| | Isopropyl myristate (IPMR) | 35.5 | 13.5 | 8.5 | 5.0 | 8.5 |
| B | Hydrogenated polyisobutene | 3.0 | 5.0 | 10.0 | 15.0 | 0 |
| | Isododecane | 0 | 0 | 0 | 0 | 10.0 |
| C | (C1) Polyglyceryl-6 | 15.0 | 15.0 | 15.0 | 15.0 | 15.0 |
| | dicaprate*¹ | | | | | |
| | (C2) Polyglyceryl-10 | 5.0 | 5.0 | 5.0 | 5.0 | 5.0 |
| | geoleate*² | | | | | |
| | Water | 0.5 | 0.5 | 0.5 | 0.5 | 0.5 |
| | Glycerin | 1.0 | 1.0 | 1.0 | 1.0 | 1.0 |
| | Total (parts by mass) | 100 | 100 | 100 | 100 | 100 |
| | (C1)/(C2) (mass ratio) | 3.0 | 3.0 | 3.0 | 3.0 | 3.0 |
| Evaluation | Makeup removal with 30% | B | A | A | A | A |
| | water-mixed cleanser | | | | | |
| | Appearance of 30% water- | A | A | A | A | A |
| | mixed cleanser | | | | | |
| | Viscosity of 30% water- | A | A | A | A | A |
| | mixed cleanser | | | | | |

TABLE 3

| Component | | Example 12 | Example 13 | Example 14 | Example 15 | Example 16 | Example 17 |
|---|---|---|---|---|---|---|---|
| A | Cetyl ethylhexanoate | 60.0 | 60.0 | 60.0 | 60.0 | 59.0 | 60.0 |
| | (CIO) | | | | | | |
| | Isopropyl myristate | 8.5 | 13.5 | 13.5 | 13.5 | 8.5 | 8.5 |
| | (IPMR) | | | | | | |
| B | Hydrogenated | 10.0 | 5.0 | 5.0 | 5.0 | 11.0 | 0 |
| | polyisobutene | | | | | | |
| | Isododecane | 0 | 0 | 0 | 0 | 0 | 10.0 |
| C | (C1) Polyglyceryl-6 | 15.0 | 17.5 | 18.0 | 18.18 | 15.0 | 15.0 |
| | dicaprate*¹ | | | | | | |
| | (C2) Polyglyceryl-10 | 5.0 | 2.5 | 2.0 | 1.82 | 0 | 5.0 |
| | geoleate*² | | | | | | |
| | (C2) Polyglyceryl-10 | 0 | 0 | 0 | 0 | 5.0 | 0 |
| | diisostearate*³ | | | | | | |
| | Water | 0.5 | 0.5 | 0.5 | 0.5 | 0.5 | 0.5 |
| | Glycerin | 1.0 | 1.0 | 1.0 | 1.0 | 1.0 | 1.0 |
| | Total (parts by mass) | 100 | 100 | 100 | 100 | 100 | 100 |
| | (C1)/(C2) (mass ratio) | 3.0 | 7.0 | 9.0 | 10.0 | 3.0 | 3.0 |
| Evaluation | Makeup removal with | A | A | A | A | A | A |
| | 30% water-mixed | | | | | | |
| | cleanser | | | | | | |
| | Appearance of 30% | A | A | A | B | A | A |
| | water-mixed cleanser | | | | | | |
| | Viscosity of 30% | A | A | A | A | A | A |
| | water-mixed cleanser | | | | | | |

*³Decaglyceryl diisostearate consisting of isostearic acid (having 18 carbon atoms) and polyglyceryl having an average polymerization degree of 10 (HLB value: 11.1)

TABLE 4

| | | Com. | Com. | Com. | Com. | Com. | Com. | Com. | Com. | Com. |
|---|---|---|---|---|---|---|---|---|---|---|
| | | Example | Example | Example | Example | Example | Example | Example | Example | Example |
| Component | | 1 | 2 | 3 | 4 | 5 | 6 | 7 | 8 | 9 |
| A | Cetyl ethylhexanoate (CIO) | 80 | 90 | 70 | 83.5 | 60 | 58.5 | 60 | 17.7 | 61.15 |
| | Isopropyl myristate (IPMR) | 0 | 0 | 0 | 0 | 20.5 | 0 | 13.5 | 0 | 8.66 |
| B | Hydrogenated polyisobutene | 0 | 0 | 0 | 0 | 3 | 0 | 5 | 0 | 10.19 |
| C | (C1) Polyglycerol-6 | 15 | 7.5 | 22.5 | 11.5 | 11.5 | 15 | 20 | 0 | 0 |
| | dicaplate*¹ | | | | | | | | | |
| | (C2) Polyglyceryl-10 | 5 | 2.5 | 7.5 | 5 | 5 | 5 | 0 | 0 | 0 |
| | geoleate² | | | | | | | | | |
| | POE(8) Glycerin | 0 | 0 | 0 | 0 | 0 | 0 | 0 | 40 | 20 |
| | monoisostearate | | | | | | | | | |
| | Mineral oil | 0 | 0 | 0 | 0 | 0 | 20 | 0 | 42.3 | 0 |
| | Water | 0 | 0 | 0 | 0 | 0 | 0.5 | 0.5 | 0 | 0 |
| | Glycerin | 0 | 0 | 0 | 0 | 0 | 1 | 1 | 0 | 0 |
| | Total (parts by mass) | 100 | 100 | 100 | 100 | 100 | 100 | 100 | 100 | 100 |
| | (C1)/(C2) (mass ratio) | 3.0 | 3.0 | 3.0 | 2.3 | 2.3 | 3.0 | — | — | — |
| Evaluation | Makeup removal with 30% | B | C | C | C | C | C | C | D | D |
| | water-mixed cleanser | | | | | | | | | |
| | Appearance of 30% water- | C | C | B | B | B | C | B | A | C |
| | mixed cleanser | | | | | | | | | |
| | Viscosity of 30% water-mixed | A | A | A | A | A | A | A | D | C |
| | cleanser | | | | | | | | | |

From the above results, it is understood that the cleanser according to the present invention does not thicken even when mixed with 30% by mass of water, has high transparency, and is excellent in the makeup-removing effect. In consideration of the results together with the results of the Comparative Examples, it is understood that for the cleanser according to the present invention, (A) an ester oil, (B) an aliphatic hydrocarbon having 8 or more and 20 or less carbon atoms, (C1) a polyglyceryl difatty acid ester consisting of a fatty acid having 8 or more and 10 or less carbon atoms and a polyglyceryl having an average polymerization degree of 4 or more and 10 or less, and (C2) a polyglyceryl difatty acid ester consisting of a fatty acid having 18 or more and 22 or less carbon atoms and a polyglyceryl having an average polymerization degree of 8 or more and 15 or less are mixed at once, and the content of (A) an ester oil and the mass ratio of components (C1) and (C2) are adjusted, thereby achieving excellent effects.

Meanwhile, it is understood that in a case in which the component (B) is not mixed, the makeup-removing effect and appearance of the 30% water-mixed cleanser deteriorate. It is also understood that in a case in which at least one of the components (C1) and (C2) is not mixed, the makeup-removing effect or appearance deteriorates. Furthermore, it is understood that even when the component (B) is mixed, if the mass ratio ((C1)/(C2)) of components (C1) and (C2) is excessively low, the makeup-removing effect and appearance of the 30% water-mixed cleanser deteriorate.

1. A transparent cleanser, comprising:
(A) an ester oil;
(B) an aliphatic hydrocarbon having 8 or more and 20 or less carbon atoms; and
(C) a nonionic surfactant,
wherein a content of the ester oil (A) is 40% by mass or more and 90% by mass or less with respect to a total amount of the transparent cleanser,
wherein the nonionic surfactant (C) comprises:
(C1) a polyglyceryl difatty acid ester consisting of a fatty acid having 8 or more and 10 or less carbon atoms and a polyglyceryl having an average polymerization degree of 4 or more and 10 or less; and
(C2) a polyglyceryl difatty acid ester consisting of a fatty acid having 18 or more and 22 or less carbon atoms and a polyglyceryl having an average polymerization degree of 8 or more and 15 or less, and
wherein a mass ratio of the components (C1) and (C2) is from 2.5:1 to 10:1.

2. The transparent cleanser according to claim 1, wherein the component (B) is at least one selected from the group consisting of hydrogenated polyisobutene having 16 or less carbon atoms, isododecane, and isohexadecane.

3. The transparent cleanser according to claim 1, wherein a content of the component (B) is 3% by mass or more and 30% by mass or less with respect to the total amount of the transparent cleanser.

4. The transparent cleanser according to claim 1, wherein a total content of the components (C1) and (C2) is 8% by mass or more and 45% by mass or less with respect to the total amount of the transparent cleanser.

5. The transparent cleanser according to claim 1, wherein the component (C1) is at least one selected from the group consisting of hexaglyceryl dicaprylate and hexaglyceryl dicaprate.

6. The transparent cleanser according to claim 1, wherein the component (C2) is at least one selected from the group consisting of decaglyceryl diisostearate and decaglyceryl dioleate.

7. The transparent cleanser according to claim 1, which has a moisture content of 5% by mass or less.

8. The transparent cleanser according to claim 1, which further comprises a moisturizer.

9. The transparent cleanser according to claim 1, which has a viscosity of 5000 mPa s or less when adding 30 parts by mass of water to 100 parts by mass of the transparent cleanser.

10. The transparent cleanser according to claim 1, which is a skin cleanser.

11. The transparent cleanser according to claim 1, which is a cleansing cosmetic.
